(19) United States
(12) Patent Application Publication
(10) Pub. No.: US 2024/0296946 A1
(43) Pub. Date: Sep. 5, 2024

(54) System and Method for Providing Multi-Platform Telemedicine

(71) Applicant: Shootu Holdings LLC, Dallas, TX (US)

(72) Inventors: Shibu Thomas, Irving, TX (US); Robert True, Irving, TX (US); Melissa Stewart, Hurst, TX (US); Stephanie Fargo, Hurst, TX (US); Tung Tran, Southlake, TX (US); Leia Oh-Spradling, Richardson, TX (US)

(21) Appl. No.: 18/566,283

(22) PCT Filed: May 31, 2022
(86) PCT No.: PCT/US2022/072660

§ 371 Date: Dec. 1, 2023

Related U.S. Application Data
(60) Provisional application No. 63/195,431, filed on Jun. 1, 2021.

(51) Int. Cl. G16H 40/67; G16H 15/00; G16H 50/20; G16H 80/00

(52) U.S. Cl. CPC G16H 40/67; G16H 15/00; G16H 50/20; G16H 80/00

(57) ABSTRACT

The present invention relates to telemedicine systems and methods capable of single platform, multi-platform and multi-device communication, via a plurality of configuration files, whereby the present telemedicine system is configurable to detect, interface and operate between and across multiple devices via configurations utilizing various operating systems, applications, hardware platforms, software platforms, or a combination thereof. The telemedicine system and method of use is directed specifically toward providing device-specific recognition and connection for the greatest flexibility, control, efficient, ergonomic and secure operation. Too, the present telemedicine invention enables both provider and patient prioritization to most equitably distribute scarce healthcare resources.

### CROSS-REFERENCE TO RELATED APPLICATIONS

U.S. Provisional Patent Application No. 63/195,431 filed on Jun. 1, 2021 PCT International Patent Application No. PCT/US2022/072660 filed May 31, 2022

### FEDERALLY SPONSORED RESEARCH OR DEVELOPMENT

Non-Applicable

### FIELD OF THE INVENTION

The present disclosure relates to telemedicine systems, generally, and more specifically to telemedicine systems capable of single platform, multi-platform and multidevice configurations whereby the present telemedicine system is configurable to identify and operate between and across multiple devices utilizing various operating systems, applications, hardware platforms, or a combination thereof.

### BACKGROUND OF THE INVENTION

Telehealth, or implementation of remote clinical services, in its most nascent form is a means of providing healthcare (e.g., provider-patient telecommunications) between a patient and healthcare provider (i.e., physician, pharmacist, nurse or educator) over long distances via telephonic and videographic means. These communications, typically taking place in different geographic locations, may take the form of evaluations, diagnosis, interventions, monitoring, education or any one of a variety of health-related services conducted between and among healthcare providers and/or a healthcare provider (or providers) and a patient. These physician-patient interactions, routinely deemed telemedicine, have been made increasingly easier and more accessible with advances in technology, which carry with them the potential benefits of convenience as well as improvements in overall patient health through improved healthcare delivery.

While the majority of data gathering and review (e.g., visual observations, medical records, vitals, labs, imaging and the like) is accomplished by the physician, accounting for the bulk of information retrieval and analysis, too the patient relies upon the same technology to input patient specific information (e.g., medical history, previous diagnosis, current complaint, insurance information and the like) as well as communicated verbally with and convey status visually to a provider. In this way, it is imperative to observe that each party not only requires compatibility between devices but also reassurances that no protected health information is compromised in the process.

Yet, above and beyond effective and protected relaying of information, certain expected advantages have become evident in long distance medicine along with the addition and/or increased recognition of other tangential benefits hereto fore unanticipated or, at a minimum, underappreciated.

Manifestly, along with aided convenience and increased sensitivity to physician and patient time, telemedicine provides a valuable barrier to the transmission of communicable disease, better assessment of patients in their home environment and increased access for non-ambulatory or immobile patients (in addition to patients lacking access to transportation or in rural settings) who otherwise face great challenges to attending “in-person” appointments.

What is more, telemedicine allows for a more equitable distribution of scarce healthcare worker resources where distance between workers and patients, and between and among healthcare workers themselves, becomes more traversable, electronically, and demonstrably more tenable when facilitated by technological advancements in remote communication. This is especially true where, in sparsely populated areas, access to healthcare services are severely limited if not entirely precluded by an inability to receive any practical medical services.

Moreover, those patients exhibiting acute needs can receive much needed immediate attention without expending precious energy or losing valuable time in receiving imminent care. By the same token, acute care physicians may be apportioned based on skill, knowledge or specialty to those patients requiring specialized and/or immediate care without requiring a practitioner's physical presence.

Too, those patients with chronic or ongoing conditions, or those patients receiving routine evaluations or requiring reoccurring or scheduled care may benefit by pre-scheduled appointments, schedulable at established intervals or times, which require little new diagnosis or evaluation and require simple maintenance appointments for continued care for which a face-to-face interaction provides little benefit over long-distance healthcare delivery. Patients, with fewer barriers to communications with practitioners, would be more likely to keep appointments and, providers, afforded the ability to schedule at limited times and in specific blocks, sometimes far in the future, would benefit from a structured appointment schedule, with fewer “no-shows”, wherein existing patients may be scheduled back-to-back, continuously, say into an AM schedule, and new patient appoints may be scheduled into a PM schedule, or vice versa—greatly increasing the ability of one practitioner, or several practitioners, to provide the greatest scheduled utility and benefit.

Additionally, and not inconsequential, family members, with a patient's permission, may find attending appointments more practical and achievable with the introduction of advanced means of long-distance communication.

As is observed, the promise of telemedicine is great, but its implementation is rife with both patient-centric reservations and technological challenges. Greater advances in the proper utilization of current and future technological advancements will inevitably lead to an ever-expanding adoption of technology in this most promising field especially in terms of lowering barriers of entry and operation.

### SUMMARY

The present disclosure achieves technical advantages as a system and method for providing single-platform and multi-platform telemedicine applications across various devices and platforms. This multi-platform telemedicine system includes a plurality of configuration files for a plurality of devices having different hardware and/or software (e.g., multi-platform devices) to allow the system to be configured to operably communicate with at least one device to provide telemedicine services.

The system is a single solution that can manage telemedicine needs for a hospital, ER, health facility or outpatient clinic space, as well can be as directed to consumer mobile devices across a wide range of available devices. The system itself can communicate with each device individually, as a device is or devices are detected, so that the system can interface with each available operating system, application, software or hardware platform both efficiently and effectively. The system can customize controls for a device that is used during a telemedicine session via one or more operable configuration files. The patient or physician can use a computer device provided by a medical facility, away from or within a medical facility (i.e., hospital or clinic), or use their own computer device in the form of a desktop computer, a mobile computer laptop, a mobile computer tablet, a smartphone, or a combination thereof. The system, uniquely, has the ability to detect the type of device a patient or physician is using during a telemedicine session (e.g., video connection between two or more devices) and then, based upon the characteristics of each specific device, uniquely cater offerings to that device providing the physician-user the most useful and efficient operation and the patient-user the greatest flexibility and control in each physician-patient interaction. In this way, the current system may be differentiated from other telemedicine systems that simply provide a video conference with a doctor on one end and a patient on the other end and supplant this rudimentary “video conferencing” system with a truly integrated “telemedicine system” capable of providing superior physician-patient interactions across various and varied systems.

In order to access and utilize traditional telemedicine systems, hospitals will typically rely upon their IT department to incorporate existing software or implement new software into a retrofitted communications network. Unlike other video conference systems, though, telemedicine suffers from an increased burden to limit and protect access to sensitive patient information (Protected Health Information—PHI) which is critical to its operation. In order to access a hospital network, software must be continuously verified and verifiably highly secured with every aspect of the data processing scrutinized for proper information security and integrity as well as optimized for peak effective functional operations. Further, when any new devices are connected to a facilities' network, the facilities' IT department needs newly engaged to ensure that the network connection, proxies, and other network and security issues are maintained to the highest degree of certainty. A provider must therefore be assured and reassured each time, before entering a physician-patient consultation, that all security protocols are observed, and all technical functionalities are optimized when encountering new patients or existing patients on a new device.

In contrast to existing systems, the present disclosure provides a standalone solution that can ensure integrity, eliminate security risks and provide optimal communications functionality across multiple devices and formats, all while providing an infrastructure for fluid communications between devices, by making, as much as is practicable, “localized” functionality by implementing a data server on the present system processor wherein sensitive information, device interactions and bi-directional communication can be set up as a local network of computer processors. To this end, the various physician and patient devices (e.g., desktops, tablets, smartphones) are each connected to local servers ensuring that no outside Internet is an absolute requirement for fluid operations although inventors realize that integrating an internet connection into the present construct is not necessarily precluded. The present disclosure therefore contemplates a “closed” implementation over a private intranet in a medical facility (e.g., a clinic, doctor's office or hospital) and another integrated open implementation via the Internet between different locations, potentially worldwide.

For example, the present system can be implemented within a medical call center acting as a “switching station” or like “conduit” with a plurality of physicians and healthcare providers, on the one hand, and a plurality of patients, on the other hand, associated with the medical call center. Using the technology of the present disclosure, these providers (e.g., physicians, pharmacist, nurses and ancillary staff) can each handle a range of medical inquiries to ultimately increase the availability and efficacy of a set of providers being able to engage a wide array of patients to provide the best care possible through expanded access to provided health services. Equally, patients gain benefits from a vastly expanded menu of qualified practitioners with which to gain more expedient and directed services.

Additionally, as many physicians increasingly express a desire for a freedom from a specific location (i.e., disfavor being tied down to particular facilities), a “mobile” physician workforce can be seen to more effectively provide health communications and interactions with patients based on selectable physician and patient criteria, regardless of physician (or patent) location, based on the quality of physicians and need of the patient as opposed to simply determined based on location and availability. Correspondingly, physicians can consult with patients on an ad hoc basis, for example, one to two hours here, three or four hours there. Physicians can pick up shifts or provide availability to answer patient questions in both rural and urban locations across a state, province or country, amendable to state and provincial laws, without regard to the physical location of a practitioner or patient. Thus, patient care can truly be based off practitioner skill and knowledge, along with practitioner availability, and patient need as opposed to provider and patient locations.

Accordingly, the ability to recognize the type of devices being used by a patient, essentially meeting the patient “where they are” spatially and technologically, is critical so that any location and technology limitations can be identified, integrated into the present system and overcome.

The system and method provided allows the dual advantages of (a) allowing physicians with particular expertise available to see patients from anywhere, (b) using any platform, thereby triaging patients so that those patients with the greatest need, or with specific medical issues, are prioritized or selectively routed to an available physician with requisite skill and/or area of specialization. The present system helps transfer the cumbersome workflow out of administrators and providers' hands and allows each to concentrate their efforts on effective physicians-patient interactions and handling of the highest volume of patients and those with the greatest need in the most efficient manner possible.

### BRIEF DESCRIPTION OF THE DRAWINGS

The present disclosure will be readily understood by the following detailed description, taken in conjunction with the accompanying drawings that illustrate, by way of example, the principles of the present disclosure. The drawings illustrate the design and utility of one or more exemplary embodiments of the present disclosure, in which like elements are referred to by like reference numbers or symbols. The objects and elements in the drawings are not necessarily drawn to scale, proportion, or precise positional relationship. Instead, emphasis is focused on illustrating the principles of the present disclosure.

FIG. 1 illustrates a system schematic, in accordance with one or more exemplary embodiments of the present disclosure;

FIG. 2 illustrates an administrative flowchart diagram, in accordance with one or more exemplary embodiments of the present disclosure;

FIG. 3 illustrates a provider flowchart diagram, in accordance with one or more exemplary embodiments of the present disclosure;

FIG. 4 illustrates a login page, in accordance with one or more exemplary embodiments of the present disclosure;

FIG. 5 illustrates an Admin Dashboard, in accordance with one or more exemplary embodiments of the present disclosure;

FIG. 6 illustrates an Add Healthcare Provider screen, in accordance with one or more exemplary embodiments of the present disclosure;

FIG. 7 illustrates an Add Device screen, in accordance with one or more exemplary embodiments of the present disclosure;

FIG. 8 illustrates an Add Client screen, in accordance with one or more exemplary embodiments of the present disclosure;

FIG. 9 illustrates a Test Page for Providers, in accordance with one or more exemplary embodiments of the present disclosure;

FIG. 10 illustrates a Provider-Populated Dashboard, in accordance with one or more exemplary embodiments of the present disclosure;

FIG. 11 illustrates a Device Idle Page, in accordance with one or more exemplary embodiments of the present disclosure;

FIG. 12 illustrates a Video Call screen, in accordance with one or more exemplary embodiments of the present disclosure;

FIG. 13 illustrates an Add Provider to Call screen, in accordance with one or more exemplary embodiments of the present disclosure;

FIG. 14 illustrates an Encounter Notes screen, in accordance with one or more exemplary embodiments of the present disclosure; and

FIG. 15 illustrates a “Telecart”, in accordance with one or more exemplary embodiments of the present disclosure.

### DETAILED DESCRIPTION

The preferred version of the disclosure presented in the following written description and the various features and advantageous details thereof, are explained more fully with reference to the non-limiting examples included in the accompanying drawings and as detailed in the description, which follows. Descriptions of well-known components have been omitted so to not unnecessarily obscure the principal features described herein. The examples used in the following description are intended to facilitate an understanding of the ways in which the disclosure can be implemented and practiced. Accordingly, these examples should not be construed as limiting the scope of the claims. And whereas the present invention and method of use are capable of several different embodiments, which can be arranged and rearranged into several functioning configurations, both physical and virtual, each may exhibit accompanying interchangeable functionalities without departing from the scope and spirit of the present application as shown and described.

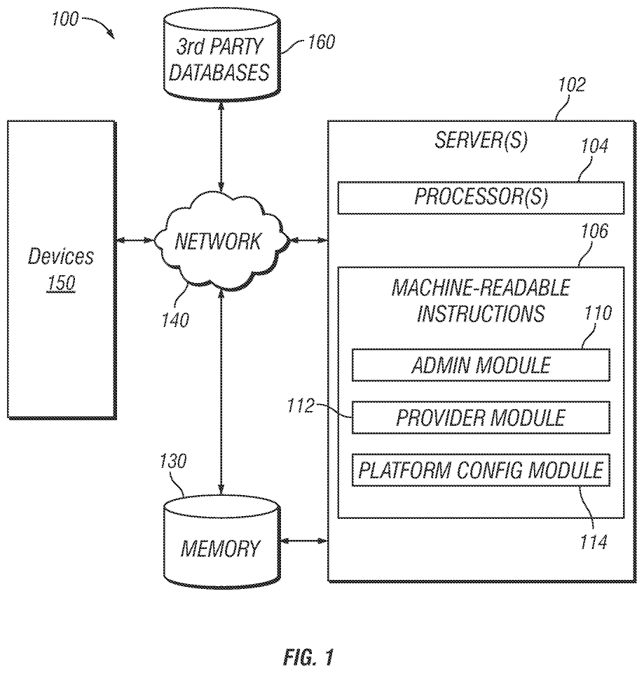

FIG. 1 illustrates a schematic view of a system 100, in accordance with one or more exemplary embodiments of the present disclosure. The system 100 may include one or more servers 102 having one or more processors 104, a memory 130, machine readable instructions 106, including an admin module 110, a provider module 112, and a platform configuration module 114, among other relevant modules. The server 102 can be operably coupled to one or more devices 150 via a network 140. The devices 150 can be a physical device (e.g., mobile phone, laptop, tablet, desktop computer, cart, wearable device, or other suitable device), program, or computer application. In another exemplary embodiment, a device 150 can include a mobile smart phone having a mobile computer application, and/or Graphic User Interface (GUI), configured to communicate or allow communication with and between the server 102 over a network 140. In other implementations of the current configuration the communications between devices 150 can be direct (i.e., without an intermediary server 102 or network device) and be conducted device-to-device. Although it is in the contemplation of inventors that both indirect (with a server or server intermediary) and direct (device-to-device) may be conducted concomitantly or alternatively as each communication interaction dictates.

In one exemplary embodiment, the admin module 110 can create a client (i.e., patient) add a provider, allow selection of a pre-loaded provider and/or add, maintain or delete a patient's device within a patient's profile. In one exemplary embodiment, a provider module 112 can display a provider-specific dashboard, directed toward a particular provider, to allow said provider to identify patients, waiting devices and online devices where a platform configuration module 114 can select a configuration file for a patient's device thus allowing the system 100 to operably communicate and control one of a plurality of makes and models of devices 150.

The aforementioned system components (e.g., server(s) 102 and device(s) 150) can be communicably coupled to each other via the network 140, such that data can be transmitted efficiently and effectively between and among devices. The network 140 can be the Internet, intranet, or other suitable network. The data transmission can be encrypted, unencrypted, over a VPN tunnel, or other suitable communication means as to protect patient health information. The network 140 can be a WAN, LAN, PAN, or other suitable network type. The network communication between the devices 150, server 102, or any other system component can be encrypted using PGP, Blowfish, Twofish, AES, 3DES, HTTPS, or other suitable encryption methods. The multi-platform telemedicine system 100 can be configured to provide communication via the various systems, components, and modules disclosed herein via an application programming interface (API), PCI, PCI-Express, ANSI-X12, Ethernet, Wi-Fi, Bluetooth, or other suitable communication protocol or medium. The system 100 can provide two-way streaming of data between a plurality of devices or between the server 102 and the devices 150, via one or more libraries, programs, or APIs (e.g., HTTP Live Streaming (HLS), Web Real-Time Communication (WebRTC), HTTP Dynamic Streaming (HDS), MPEG-DASH, etc.) Additionally, third party systems and databases can be operably coupled to the system components via the network 140. Too, patent-practitioner data may consist of “real time” communications, prerecorded communications, “real time” heath data and pre-loaded or archived health data all of which may be selectable or accessed in “real time”, prospectively or retrospectively by a practitioner, provider, medical staff, administrator or other party authorized to access such information.

The data transmitted to and from the components of multi-platform telemedicine system 100 (e.g., the server 102 and devices 150), can include any format, including JavaScript Object Notation (JSON), TCP/IP, XML, HTML, ASCII, SMS, CSV, representational state transfer (REST), or other suitable format. The data transmission can include a message, flag, header, header properties, metadata, and/or a body, or be encapsulated and packetized by any suitable format having same including scanned data, form data, packaged data, results data, provider or physician entered data, patient entered data, image data, lab data, genomic data, previous history data and the like.

The server(s) 102 can be implemented in hardware, software, or a suitable combination of hardware and software therefor, and may comprise one or more software systems operating on one or more servers, having one or more processors 104, with access to memory 130. Server(s) 102 can include electronic storage, one or more processors, and/or other components. Server(s) 102 can include communication lines, connections, and/or ports to enable the exchange of information via a network 140 and/or other computing platforms. Server(s) 102 can also include a plurality of hardware, software, and/or firmware components operating together to provide the functionality attributed herein to server(s) 102. For example, server(s) 102 can be implemented by a cloud of computing platforms operating together as server(s) 102, including Software-as-a-Service (SaaS) and Platform-as-a-Service (PaaS) functionality. Additionally, the server(s) 102 can include memory 130.

Memory 130 can comprise electronic storage that can include non-transitory storage media that electronically stores information. The electronic storage media of electronic storage can include one or both of system storage that can be provided integrally (e.g., substantially non-removable) with server(s) 102 and/or removable storage that can be removably connectable to server(s) 102 via, for example, a port (e.g., a USB port, a firewire port, etc.) or a drive (e.g., a disk drive, etc.). Electronic storage may include one or more of optically-readable storage media (e.g., optical disks, etc.), magnetically-readable storage media (e.g., magnetic tape, magnetic hard drive, floppy drive, etc.), electrical-charge-based storage media (e.g., EEPROM, RAM, etc.), solid-state storage media (e.g., flash drive, etc.), and/or other electronically-readable storage media. Electronic storage may include one or more virtual storage resources (e.g., cloud storage, a virtual private network, and/or other virtual storage resources). The electronic storage can include a database, or public or private distributed ledger (e.g., blockchain) to both efficiently store, access and protect data. Electronic storage can store machine-readable instructions 106, software algorithms, control logic, data generated by processor(s), data received from server(s), data received from computing platform(s), and/or other data that can enable server(s) to function as described herein. The electronic storage can also include third-party databases accessible via the network 140. Electronic storage may also be used as a repository for patient and provider identifying and tracking information, provider credentialling information, provider licensure information, patient health and medical history information, patient medical history information, patient lab, images and prescription history, or a combination thereof.

Processor(s) 104 can be configured to provide data processing capabilities in server(s) 102. As such, processor(s) 104 can include one or more of a digital processor, an analog processor, a digital circuit designed to process information, an analog circuit designed to process information, a state machine, and/or other mechanisms for electronically processing information, such as FPGAs or ASICs. The processor(s) 104 can be a single entity or include a plurality of processing units. These processing units can be physically located within the same device or processor 104, or across multiple devices or processor(s) 104, which can represent processing functionality of a plurality of devices or software functionality operating alone, or in concert, sequentially or simultaneously.

Processor(s) 104 can be configured to execute machine-readable instructions 106 or machine learning modules via software, hardware, firmware, some combination of software, hardware, and/or firmware, and/or other mechanisms for configuring processing capabilities on processor(s) 104. As used herein, the term “machine-readable instructions” can refer to any component or set of components that perform the functionality attributed to the machine-readable instructions component 106. This can include one or more physical processors 104 during execution of processor-readable instructions, the processor-readable instructions, circuitry, hardware, storage media, or any other components.

Server(s) 102 can be configured with machine-readable instructions having one or more functional modules. The machine-readable instructions 106 can be implemented on one or more servers 102, having one or more processors 104, with access to memory 130. The machine-readable instructions 106 can be a single networked node, or a machine cluster, which can include a distributed architecture of a plurality of networked nodes. The machine-readable instructions 106 can include control logic for implementing various functionality, as described in more detail below. The machine-readable instructions 106 can include certain functionality associated with the multi-platform telemedicine system 100. Additionally, the machine-readable instructions 106 can include a smart contract or multi-signature contract that can process, read, and write data to the database, distributed ledger, or blockchain.

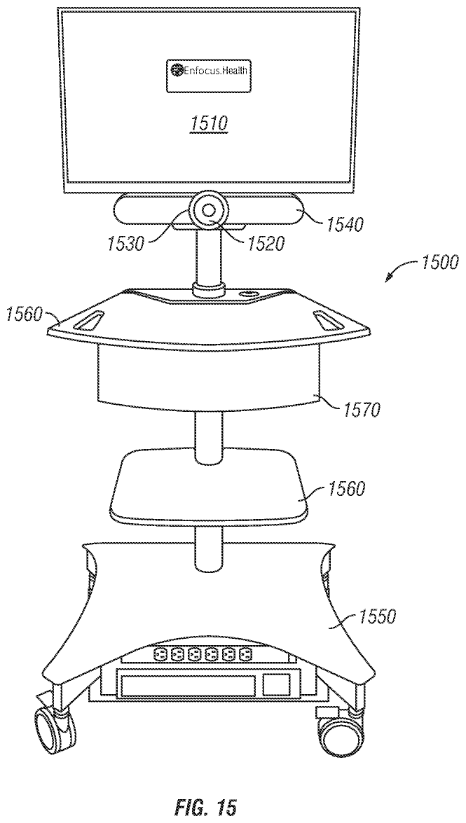

In one exemplary embodiment, the system 100 can include a device 150 operably coupled to a controller (e.g., Raspberry Pi, processor, FPGA, etc.), a display, a camera, a microphone, speakers, and a user input device. Said controller, display, camera, microphone, speaker, and input device can be integrated into the device 150 (e.g., mobile phone, tablet, laptop, etc.), one or more of the controller, display, camera, microphone, speaker, and input device can be external to a device 150, or combination of controller, display, camera, microphone, speaker, and input device may be interchangeably internal and external to a device 150. For example, the device can be a telemedicine cart (i.e., “telecart” 1500 in FIG. 15), mobile phone, tablet, or other suitable device which, in operation, can remotely accessed by an operator to control the client's camera (e.g., pan, tilt, zoom, etc.) and communicate with the patient to remotely provide medical services to the patient where the operator may not be physically present in the room with the patient or even located in the same facility as the patient.

In another embodiment, the system 100 itself, in addition to manipulating several device inputs and outputs, can internally escalate or prioritize a user session (for medical services) based at least in part on an assessment determined from a preliminary or subsequent examination. For example, a nurse or doctor can initially assess a patient during a physical or remote examination to determine the severity of the medical issue and assign the patient a priority level in the system 100 based upon the examination. Additionally, a second, third or other successive follow-on examination may provide additional information whereby a prior diagnosis may be superseded or augmented in terms of priority depending on presentation, patient state progression, improvement or worsening of symptoms. This additional information may be utilized to either raise, lower or confirm an original prioritization.

In another exemplary embodiment, the provider module can provide a user interface to input patient related data, including presentation, patient history, patient medications, patient allergies, a priority level or a combination thereof. Additionally, the server can be implemented on a controller on a device to form a private network not requiring Internet access. The server implementation on the controller allows the system 100 to be deployed anywhere within a facility and allow devices to access the controller, which can handle all relays or all the security list connections to set up a local intranet. Said intranet may be centralized or decentralized and existing on one to a plurality of servers across a network 140.

In another exemplary embodiment, the ‘admin dashboard’ can be accessed via a ‘log in’ screen and can include a listing of clients (i.e., patients), client devices, providers and provider devices. The system 100 can receive user input via a “Dictaphone”, microphone, keyboard, mouse, or other suitable input device. The system 100 can create a client, such as an entity or individual, among other suitable client types wherein once the client profile is created via the system 100, data related to the client can be stored in a client database and associated with a specific client. Providers and devices can be associated with the client, provider, each or both, by adding metadata to the client database related to the providers, devices, or other relevant information. Metadata can include unique identifiers, timestamps, and other relevant data which may be specific to a particular client. Moreover, data may be encrypted or otherwise encoded as to provide another level of security for PHI (e.g., via blockchain, public-key (asymmetric) cryptography, symmetric key encryption or the like) wherein plaintext is converted into ciphertext which is only discernable to those parties with prerequisite authorization.

In yet another exemplary embodiment, the system 100 can add multi-platform devices to a client. For example, the system 100 can add a “Telecart” 1500, a tablet, a desktop, a laptop, a mobile phone, or any other suitable hardware or software that is detected or preidentified as a device and associated said device or devices with a client or client's device.

In another exemplary embodiment, the system 100 can access one or more configuration files associated with a particular device. The configuration files can include driver information, operating system information, browser information, video information, network information, database information, API information, or other relevant information.

In one exemplary embodiment, the configuration file can be a shell script, an executable file, a data file in csv, xml, txt, or other suitable format. The system 100 can prompt a user (e.g., patient, practitioner, etc.) to select the type of device that will be used during a particular telemedicine session. Upon receipt of the device selection, the system 100 can retrieve the appropriate configuration file from memory or retrieve and download a configuration file as need arises or updates dictate. The system 100 can then execute code to configure and customize the system 100 to utilize the selected device. The system 100 can further configure the session settings to suit the selected device. For example, the system 100 can set the screen resolution, camera controls, networks settings, and video codecs, among other relevant settings as to conform to a particular device or across devices.

In another exemplary embodiment, the system 100 can autoload settings or files, such as the drivers for the selected device or provide controls to the user so the user can control that device, in an asynchronous manner. If, for example, the device is a tablet, the system 100 can detect gestures or utilize the controller panel, using data from the configuration file. If, however, the device is a desktop computer, then the system 100 can configure and recognize hot keys/buttons so that keyboard arrows can be used to control the camera, among other controllable items. Advantageously, the system 100 can set up the environment and the controls for the selected device by detecting the device's capabilities and inputs.

In another exemplary embodiment, the system 100 can prompt the user of a device to download or share a link to a script. The script can determine the device settings and transmit them to the system 100. The system 100 can then receive the settings configuration to control the actuators that can move the camera to pan and tilt the camera, as well as zoom and focus the camera.

In another exemplary embodiment, the system 100 can select the configuration files/scripts that allow interaction with the selected device and can receive metadata related to the selected device (via user entry or script execution), so the location of the selected device is known. The selected device can be provided by the medical facility or by the patient. The system 100 can be configured to additionally add or allow access to one or more providers to a client, one or more clients to a provider or providers, add providers to providers (in terms of consolations and like interactions), and/or clients to clients (in terms of support groups). Said access may be based on invites, permissions, authorizations or a combination thereof initiated.

In another exemplary embodiment, after a new device is detected and established, all the device information can be used by the physician from the client device so that the physician can control the patient device with a patient's revokable permissions.

In another exemplary embodiment, the system 100 can display the online devices, inactive devices, or a combination thereof in an admin dashboard. A connect button can be displayed proximate each online device so the physician can connect to the device and start a telemedicine session.

In another exemplary embodiment, control buttons and icons can be displayed on a physician's device, a patient's device, or both devices connected during a telemedicine session. For example, the system 100 can display icons and buttons, on one or each device, that can mute audio or video, as well as move the camera right, left, up, down, zoom in, zoom out, among other options.

In another exemplary embodiment, the session can be between a patient device and a physician's device and the controls may only be present on the physician device so only the physician can control the patient device, with or without authorizations and permissions. For example, the physician device can control a camera's digital zoom or can ‘zoom in’ on video image digitally or an image can be ‘zoomed in’ on digitally (via image processing) or physically (via camera lens manipulation) and the video image can be marked, annotated, and/or saved as data for storage or later retrieval and review. For example, the camera on a phone or tablet can be controlled or manipulated via a system API.

In another exemplary embodiment, the system 100 can include a waiting room where each waiting device enters a queuing system where the provider can select a particular patient to consult and a patient may be triaged based on selected or selectable criteria or a combination thereof. For example, a patient may achieve a certain preference or hierarchy based on (1) a physician or physicians (e.g., specialization of a physician, skill of a physician, experience of a physician, primary and secondary language of a physician, seniority of a physician, and/or availability of a physician), (2) a patient (e.g., number of patients, nature of injury or illness, type of patient injury or illness, patient presentation, severity of presentation, criticality of an injury or illness, information gathered at intake and the like) or (3) criteria related to both physician and patients (e.g., type of patient-physician interaction, ratio of patients to physicians, patient presentation and condition in relation to availability and skill of physicians, or a combination thereof). It should be noted that several criteria may be weighted and weighed in relation to other variables, via, for example, an algorithmic computation, intuitive logic or artificial (machine) learning wherein weights and values may be based on and in relation to the weight or value assigned to each or other variables.

In yet another exemplary embodiment, priority may be established or patients triaged by the present system 100 whereby certain criteria may be selected or selectable by (1) an input operator (e.g., intake personnel), (2) by the system itself, or (3) by the physician, based on selectable or preprogrammed criteria or other inputted information, or a combination thereof.

In another exemplary embodiment, a provider (e.g., physician, nurse, nurse practitioner or administrator) can select a patient to start a telemedicine session with presentation information, patient history, or a combination thereof, such as vitals information, medical history, insurance information, payment information, or enrollment information, among others. Based upon this information, the system can provide triaging or assignment of patients to those physicians with a particular specialty, skill, tenure or availability. Similarly, a provider may be selectable by a patient wherein certain provider skills or knowledge may be accessible to a patient wherein a set, or subset of physicians, may be selected or selectable by a patient for a given interaction. Moreover, a patient may be identified as an existing or established patient of a certain physician, or, conversely, as a patient having no previous relationship with a physician as to assign preference or status in a queue. Too, a patient which has previously abused a particular patient-physician relationship may be deselected or removed from a particular physician's queue as a precautionary or protective measure by the system or a specific physician. Alternatively, a provider may also be ‘deselected’ or removed by a patient not wishing to interact with a particular provider (or providers).

In another embodiment, the system can auto-connect patient with physicians on a “first come, first served” basis on the side of the patient, physician or a combination thereof.

In another exemplary embodiment, the patient may register at a “receptionist counter”, representationally, and initiate an initial check-in process through, for example, a tablet. The system 100 can then add the user to push notifications to an admin dashboard and each time, for example, a tablet enters the wait list the system 100, said system 100 can generate a notification to inform the physician that a patient is waiting, the patient condition, patient history, signs and symptoms upon presentation, severity of illness or injury, vitals, or a combination thereof.

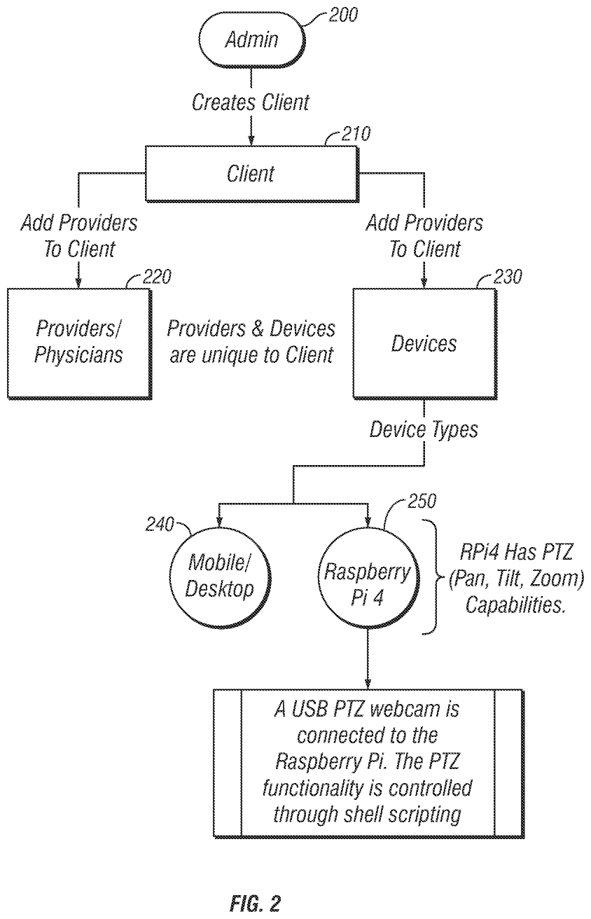

FIG. 2 illustrates a flow chart diagram exemplifying control logic embodying features of a method and system for administrative telemedicine processing, in accordance with one exemplary embodiment of the present disclosure. The administrative telemedicine processing control logic can be implemented as an algorithm on a server, a machine learning module, or other suitable system with which to implement the present invention. The administrative telemedicine processing control logic can be achieved with software, hardware, an application programming interface (API), a network connection, a network transfer protocol, HTML, DHTML, JavaScript, Dojo, Ruby, Rails, other suitable applications, or a suitable combination thereof.

The administrative telemedicine processing control logic can leverage the ability of a computer platform to spawn multiple processes and threads by processing several data sets simultaneously. The speed and efficiency of the administrative telemedicine processing control logic can be greatly improved by instantiating more than one process to facilitate administrative telemedicine processing. However, one skilled in the art of programming will appreciate that use of a single processing thread may also be utilized and is within the scope of the present invention.

The administrative telemedicine processing control logic process can be seen in FIG. 2 whereby Admin 200 (corresponding to admin module 110 in FIG. 1) creates Client 210 to which is assigned a Provider/Physician 220 and assigned “Device” 230 (corresponding to Devices 150 in FIG. 1) wherein in FIG. 2 Provider/Physician 220 and “Device” 230 is unique to each “Client” 230. Each device 150 type, Mobile/Desktop 240 and Raspberry Pi4 250, may be configurable or configured with PTZ capability to include pan, tilt, zoom among other functionalities.

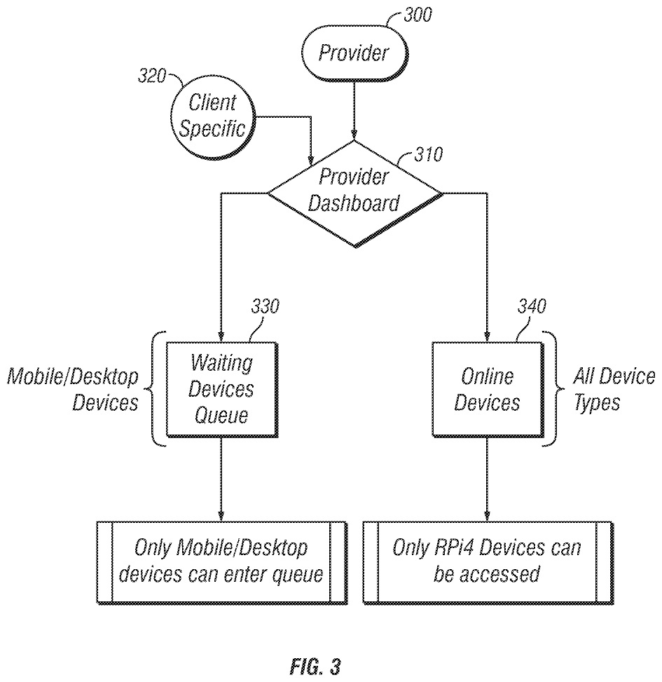

FIG. 3 illustrates a flow chart diagram exemplifying control logic embodying features of a method for provider telemedicine processing, in accordance with one exemplary embodiment of the present disclosure. The provider telemedicine processing control logic can be implemented as an algorithm on a server, a machine learning module, or other suitable system. The provider telemedicine processing control logic can be achieved with software, hardware, an application programming interface (API), a network connection, a network transfer protocol, HTML, DHTML, JavaScript, Dojo, Ruby, Rails, other suitable applications, or a suitable combination thereof.

The provider telemedicine processing control logic can leverage the ability of a computer platform to spawn multiple processes and threads by processing data simultaneously. The speed and efficiency of the provider telemedicine processing control logic can be greatly improved by instantiating more than one process to facilitate provider telemedicine processing. However, one skilled in the art of programming will appreciate that use of a single processing thread may also be utilized and is within the scope of the present invention.

The provider telemedicine processing control logic process can be seen in FIG. 3 whereby specific “Provider” 300 accesses provider dashboard 310, via a provider module 112 (see FIG. 1) where specific “Clients” 320 appear in a list or grouping in order of arrival or tiered into a waiting queue, based on various preselected or selectable criteria (described above), which are further subdivided into a waiting device queue 330 for mobile devices, desktop devices, or both or online device queue 340. Waiting device queue 330 can only be accessed and populated by mobile and desktop devices wherein online devices 340 can only be accessed by Raspberry Pi (ex. RPi4) devices. After a device has been selected, the physician/operator can initiate the provider-patient communication using WebRTC (Web Real-Time Communication) technology for video teleconferencing.

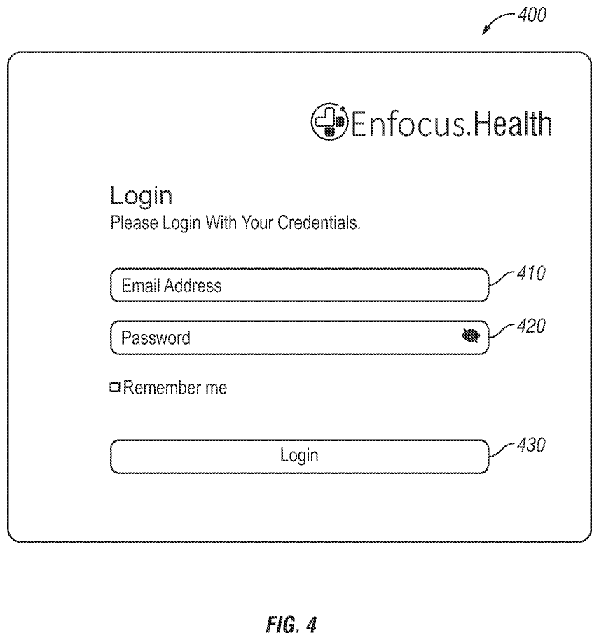

FIG. 4 illustrates a login page 400 of system 100, in accordance with one or more exemplary embodiments of the present disclosure wherein a system 100 user (e.g., provider, operator or administrator) provides a user ‘log in’ 410 and password 420 wherein said user login 410 is exemplified as an email address and access is granted through the logon icon 430.

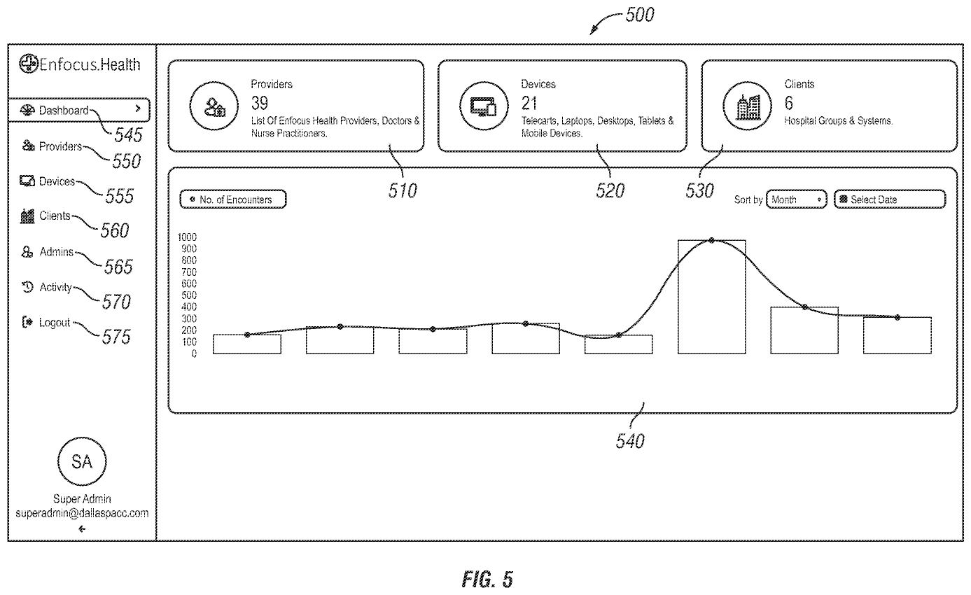

FIG. 5 illustrates an ‘Admin Dashboard’ 500 of system 100, in accordance with one or more exemplary embodiments of the present disclosure where said ‘Admin Dashboard’ 500 is further subdivided into a providers queue 510, a devices queue 520 and a clients queue 530. The providers queue 510 consists of a list of available providers, devices queue 520 consists of devices (ex. “Telecarts” 1500, laptops, desktops, tablets and mobile devices) and clients queue 530 consisting of hospital groups and systems. Monthly activity 540 (i.e., number of monthly encounters) is further displayed beneath said providers queue 510, a devices queue 520 and a clients queue 530. What is more, a provider or system operator may access each of the various subsections 510, 520 and 530 via the various icons dashboard 545, providers 550, devices 555, clients 560, admins 565, activity 570 and logout 575.

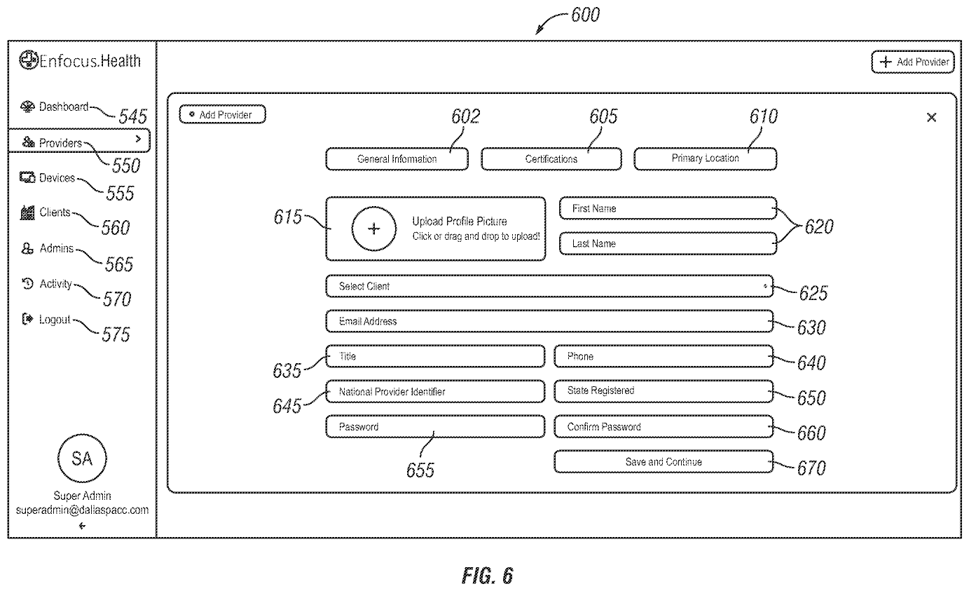

FIG. 6 illustrates an ‘Add Healthcare Provider’ screen 600 of system 100 accessible by ‘Providers’ icon 550 in FIG. 5, in accordance with one or more exemplary embodiments of the present disclosure allows for providers to indicate, through actuation of an icon, ‘General Information’ 602, ‘Certifications’ 605, and Primary Location 610 whereby a provider may ‘Upload a Profile Picture’ 615, add identifying information 620 (e.g., first and last name), designate and/or select a patient through ‘Select Client’ 625, add ‘email’ 630, add ‘Title’ 635 and ‘Phone’ 640 as well as National Provider Identifier (NPI) number 645, State Registration 650, Password 655 and ‘Confirm Password’ 660 and a final icon to ‘Save and Continue’ 670. Information may be entered by selecting a corresponding icon, as above, or via a ‘drop down’ menu, or a combination thereof.

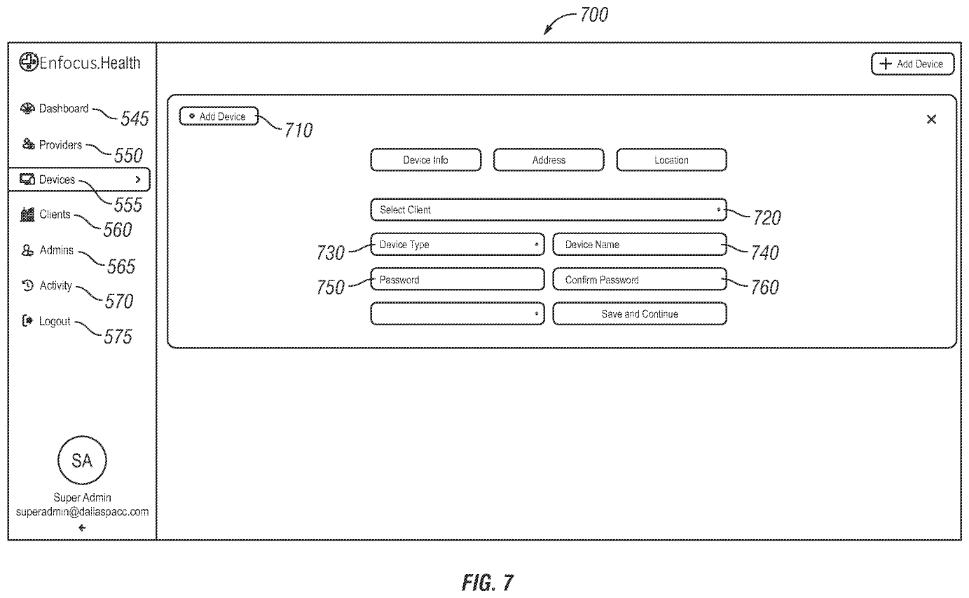

FIG. 7 illustrates an ‘Add Device’ screen 700 of system 100, in accordance with one or more exemplary embodiments of the present disclosure wherein a device may be added to system 100 by selecting ‘Device’ icon 555 and ‘Add Device’ icon 710 allowing access to select a client via ‘Select Client’ icon 720 allowing the system 100 operator to select a device type and device name via ‘Device Type’ icon 730 and Device Name′ icon 740, respectively. The user is them promoted to add their password, via ‘Password’ icon 750 and to reenter password via ‘Confirm Password’ icon 760, which may be selectable as an icon or dropdown menu as above. Information may thereafter be stored and saved into the present system 100 by selecting the ‘Save and Continue’ icon 770 once all information has been selected and/or entered.

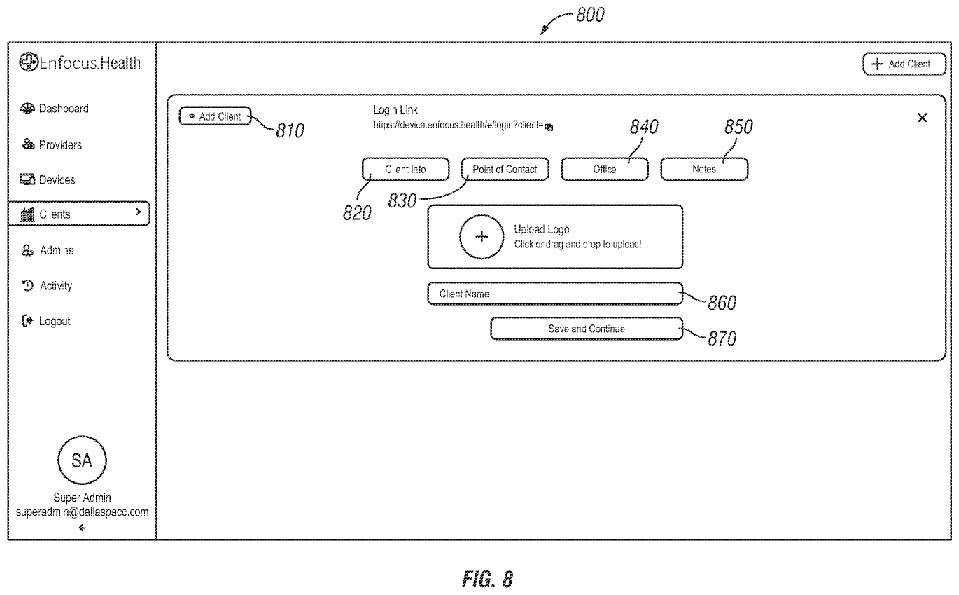

FIG. 8 illustrates an ‘Add Client’ screen 800, in accordance with one or more exemplary embodiments of the present disclosure wherein client (e.g., patient0 may be added or selected by adding client, through the ‘Add Client’ icon 810 and client info via the ‘Client Info’ tab 820 whereby point of contact, office and notes may be selected or entered via each of a ‘Point of Contact’ icon/tab 830, ‘Office’ icon/tab 840, and ‘Notes’ icon or tab 850 whereby a client name will appear in the ‘Client Name 860 indicated field 860 followed by accessing the ‘Save and Continue’ icon 870 for storing and saving information.

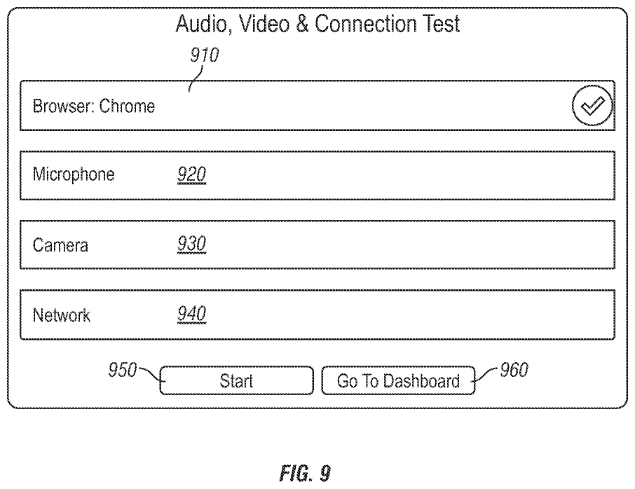

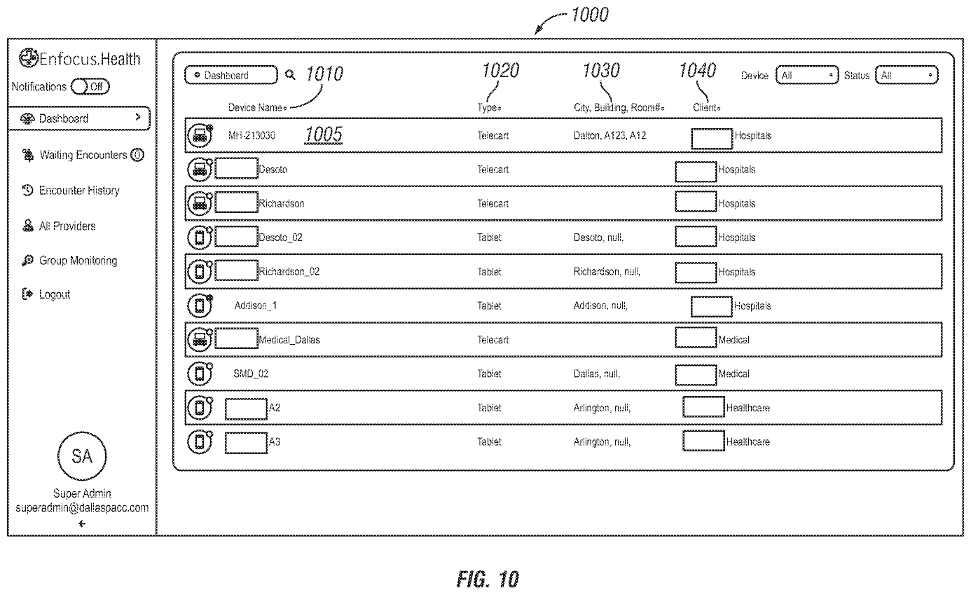

FIG. 9 illustrates a ‘Test Page’ 900 for Providers, in accordance with one or more exemplary embodiments of the present disclosure wherein, once a browser is confirmed 910, a videographic session's components may be first tested, confirming microphone 920, camera 930 and network connection 940, and then the session is initiated through ‘Start’ icon 950 wherein Dashboard 500 remains accessible through the ‘Go To Dashboards’ icon (now populated Dashboard 1000 as in FIG. 10) giving accumulated information relating to ‘Device Name’ list 1010, as well as related activity, ‘Device Type’ list 1020, location information 1030 and client 1040.

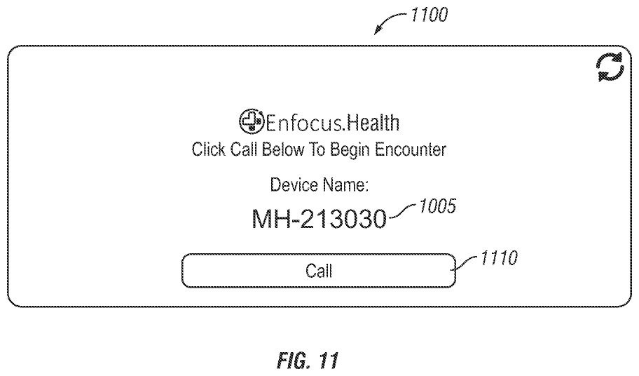

Once a device is selected, here device MH-213030 1005, a provider is allowed to open a session with a patient wherein FIG. 11 illustrates a ‘Device Idle’ page 1100 (see FIG. 11) prompting the user/provider to engage the patient through accessing the ‘Call’ icon 1110.

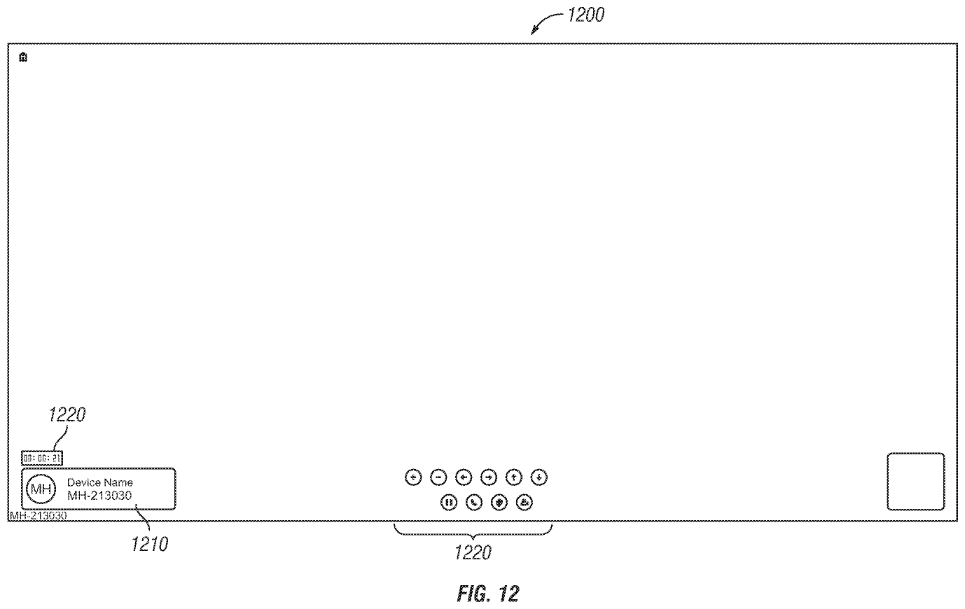

FIG. 12 illustrates a Video Call screen 1200, wherein the provider/patient interaction is conducted. As is evident from FIG. 12, the ‘Device Name’ icon 1210, signifying MH-213030 1005 and specific to the current session, appears in the lower left of the Video Call screen 1200 accompanied by the duration of the session 1220, but this may appear in various areas but not that would obscure the patient. Moreover, the provider has the options of controlling the session virtually via controls 1220 (said controls increasing zoom (+), decreasing zoom (−) and controlling operation, through left side (←), up (↑), right side (→), and down (↓) icons indication as well as icons for pause, end, mute and ‘add person’ functions).

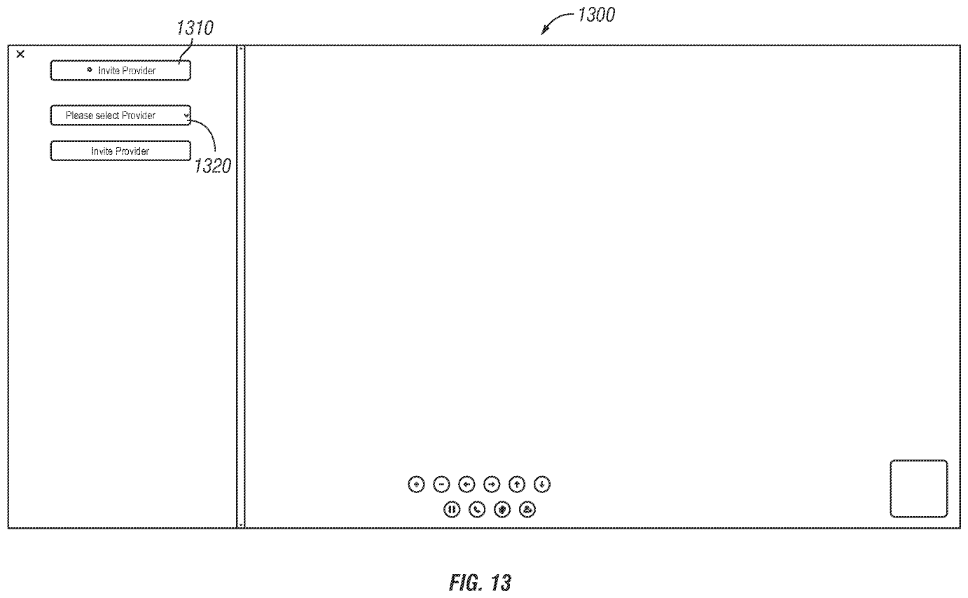

FIG. 13 illustrates an ‘Add Provider to Call’ screen 1300, in accordance with one or more exemplary embodiments of the present disclosure wherein a provider, that is an existing member or participant in the system 100, may be added to a session, via an ‘Invite Provider’ icon 1310. Said ‘Invite Provider’ icon 1310 may then allow access to a list of providers, here evidenced as a dropdown menu 1320. Alternatively, a provider may be granted temporary access or sent a prompt, via email, text message and the like, to either permanently joining a list of providers or be granted temporary access.

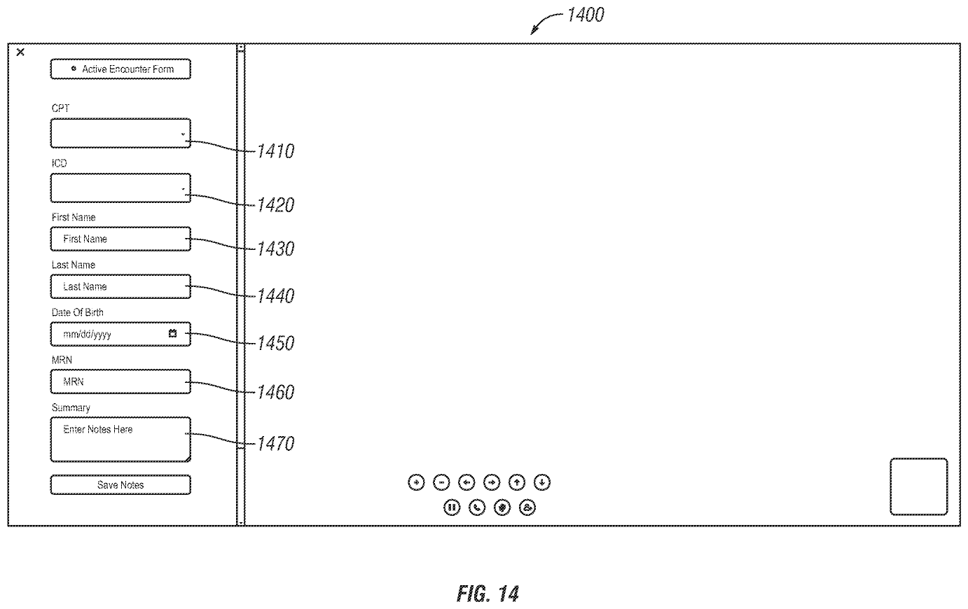

FIG. 14 illustrates an ‘ Active Encounter Form’ screen 1400, in accordance with one or more exemplary embodiments of the present disclosure, where CPT (Current Procedural Technology) 1410 may be fillable or selectable, ICD (International Statistical Classification of Diseases and Related Health Problems) 1420 may be fillable or selectable, patient first name 1430 and last name 1440, may be fillable, and DOB 1450 may be fillable or selectable. Further, MRN (Medical Record Number) 1460 may be fillable or selectable and notes and or summary 1470 may be fillable as to document the session and/or encounter before, during or after consultation and/or session.

FIG. 15 illustrates a “Telecart” 1500, in accordance with one or more exemplary embodiments of the present disclosure. The “Telecart” 1500 can include a controller, a display 1510, a camera 1520, a microphone 1530, a speaker 1540, a keyboard, a mouse, a rolling base 1550, one or more shelves 1560, and one or more storage areas 1570, among other components. In another exemplary embodiment, the controller can be a Raspberry Pi computer that exemplifies a comprehensive standalone telecommunications unit that may be preemptively stored in a hospital, office or medical facility and made portable/mobile, via said rolling base 1550, to accommodate long distance teleconferencing between designated for intrafacility or interfacility consultations and interactions where the “Telecart” 1500 may include all components of a computer, table or smartphone including configurations for Wi-Fi® and Bluetooth® wireless communications. Pointedly, the “Telecart” 1500 may be utilized where a provider is offsite (or onsite, but in another area of a campus) and the patient is disposed in another part of a designated facility. Too, this “Telecart” 1500 may be used for communications between consulting physicians, either offsite or onsite, and or group consultations, group sessions or for educational purposes with participants either onsite or offsite.

The description in this disclosure should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f).

The disclosure may be embodied in other specific forms without departing from the spirit or essential characteristics thereof. For example, each of the new structures described herein, may be modified to support particular variations or requirements while retaining their basic configurations or structural relationships with each other or while performing the same or similar functions described herein.

The present embodiments are therefore to be considered in all respects as illustrative and not restrictive. Accordingly, the scope of the disclosure can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

Too, while the invention itself and method of use are amendable to various modifications and alternative configurations, specific embodiments thereof have been shown by way of example in the drawings and are herein described in adequate detail to teach those having skill in the art how to make and practice same. It should, however, be understood that the above description and preferred embodiments disclosed, are not intended to limit the invention to the particular embodiment disclosed, but on the contrary, the present disclosure is intended to cover all modifications, alternatives and equivalents falling within the spirit and scope of the invention as defined within the claim's broadest reasonable interpretation that is consistent with the specification.

What is claimed is:

1. A system for providing single-platform and multi-platform telemedicine communications across and between various devices comprising:
one to a plurality of servers;
one to a plurality of processors;
a memory;
said memory being integral, removable or both integral and removable;
said memory including a database, a public or private distributed ledger, or both;
machine readable instructions comprising:
an admin module, a platform configuration module, a provider module, and a patient module;
said admin module managing said provider module, said patient module and a patient's device module;
said configuration module selecting a configuration file for a patient's device;
said provider module exhibiting a provider-specific module displaying a provider-specific dashboard indicating both selectable patients and patient devices;
said patient module providing information comprising identifying information, tracking information, MRN, ICD codes, medical history, previous diagnoses, current complaint, insurance information, payment information, patient labs, patient prescription history, patient images, and enrollment information;
said device module capable of recognizing multi-platform devices detectable and accessible by one or more configuration files;
said platform configuration module incorporating said configuration files; and
a communication device or devices.

2. The system of claim 1 for providing single-platform and multi-platform telemedicine communications across and between various devices wherein said devices may be a physical a device: a mobile phone, laptop, tablet, desktop computer, a telecart, wearable device, or other suitable device, a program or computer application.

3. The system of claim 1 wherein said configuration file is a shell script, an executable file, or a data file which may include driver information, operating system information, browser information, video information, network information, database information, API information, or other relevant information, which is accessed from memory or retrieved and downloaded to a device.

4. The system of claim 1 wherein system configuration files are actuatable by executing code to configure and customize said configuration files compatible with said patient's device.

5. The system of claim 4 wherein said system can prompt a user of a device to download or share a link to a script whereby the script can determine the device settings and transmit them to the system wherein configuration or customization is based on device type and model.

6. The system of claim 2 for providing single-platform and multi-platform telemedicine communications across and between various devices wherein a said computer application is accessible via a graphic user interface.

7. The system of claim 1 for providing single-platform and multi-platform telemedicine communications across and between various devices wherein said server or servers may be operably coupled to one or more devices via a network or information may be conveyed directly between devices.

8. The system of claim 7 wherein said network is an internet, an intranet or a combination thereof for transmission of information either encrypted, unencrypted or a combination of encrypted and unencrypted information.

9. The system of claim 1 wherein said device is operably coupled to a controller, a display, a camera, a microphone, speakers, and a user input device which is controllable by the system, a user and a physician, remotely.

10. The system of claim 1 wherein a provider dashboard may display patients, a controller and patient devices to allow said provider to identify and prioritize patients based on number of patients, type of patients, number of providers, types of providers, time of registration, presentation, examination, an additional examination, a patient's state progression, a patient's symptoms worsening or improvement, urgency, acuity or a combination thereof.

11. A method for conducting a telemedicine session via the system of claim 1, between a provider and patient, via the following steps comprising:
entering by a provider a password protected provider admin module;
logging into said admin module having a provider dashboard;
said dashboard having a list of providers, a list of patients together with patient-specific devices, and faculty clients;
accessing a patient waiting device queue, an online device queue, or a combination thereof, of preidentified and configured patient devices;
said waiting device queue consisting of mobile and desktop devices;
said online devices consisting of RPi4 devices;
said waiting and online devices further designated by a client facility;
accessing one of said waiting devices, online devices and client facilities;
choosing a patient with an associated configured patient device; and
conducting a telemedicine session.

12. The method of claim 11 wherein physician number, availability and skill and patient number, availability and condition severity may be ranked, separately or in combination, via selectable criteria via algorithmic computation, intuitive logic or artificial (machine) learning wherein weights and values may be based on and in relation to the weight or value assigned to each or selected variables.

13. The method of claim 12 wherein said waiting device queue, an online device queue, or a combination thereof, of preidentified and configured patient devices may be prioritized and tiered according to said selectable criteria including: number of queued patients, type of queued patients, number of queued providers, types of queued providers, time of queued patient registration, patient presentation, patient examination, patient history, an additional examination, a patient's state progression, a patient's symptoms worsening or improvement, urgency, acuteness of injury or illness, chronic nature of injury or illness, or a combination thereof, for provider selection of patient.

14. The method of claim 12 wherein said waiting device queue, an online device queue, or a combination thereof, of preidentified and configured patient devices may be prioritized and tiered according to said selectable criteria including: number of queued patients, type of queued patients, number of queued providers, types of queued providers, time of patient registration, patient presentation, patient examination, an additional patient examination, a patient's state progression, a patient's symptoms worsening or improvement, urgency, acuteness of injury or illness, chronic nature of injury or illness, or a combination thereof, for patient selection of provider.

15. The method of claim 12 wherein said waiting device queue, an online device queue, or a combination thereof, of preidentified and configured patient devices may be prioritized and tiered according to said selectable criteria including: number of queued patients, type of queued patients, number of queued providers, types of queued providers, time of patient registration, patient presentation, patient examination, an additional patient examination, a patient's state progression, a patient's symptoms worsening or improvement, urgency, acuteness of injury or illness, chronic nature of injury or illness, or a combination thereof, for patient selection of provider, provider selection of patient or a combination thereof.

16. The method of claim 12 wherein priority may be given, patients may be selected, patients may not be selected, or patients may be triaged by intake personnel, automatically via preset criteria or by the provider.

17. The method of claim 12 wherein said patients may make selection or non-selection of providers based on number, availability, knowledge, skill, or a combination thereof.

18. The method of claim 12 wherein said provider may control device settings remotely.

19. The method of claim 12 wherein a provider may conduct sessions with patients, other providers or a combination of patients and providers.
